(19) United States
(12) Patent Application Publication
(10) Pub. No.: US 2005/0234530 A1
(43) Pub. Date: Oct. 20, 2005

(54) Apparatus for hyperthermia treatment and abnormality notification method thereof

(75) Inventors: Tomoyuki Takashino, Hino-shi (JP); Taisuke Sato, Hachioji-si (JP); Masanobu Yamamoto, Fuji-shi (JP); Shigeki Ariura, Ebina-shi (JP); Akira Sakaguchi, Minami-Ashigara-city (JP); Yuuichirou Irisawa, Fujisawa-shi (JP); Wataru Karino, Princeton, NJ (US); Seiichi Ootsubo, Ashigarakami-gun (JP)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignees: OLYMPUS CORPORATION, Tokyo (JP); TERUMO KABUSHIKI KAISHA, Tokyo (JP)

(21) Appl. No.: 11/094,577

(22) Filed: Mar. 31, 2005

(30) Foreign Application Priority Data
Mar. 31, 2004 (JP) 2004-107667

(51) Int. Cl. A61F 7/00; A61F 7/12

(52) U.S. Cl. 607/89; 607/96; 607/113

(57) ABSTRACT

It is provided an apparatus for hyperthermia treatment capable of detecting emptiness of the storage unit or leakage of the cooling fluid. The apparatus for hyperthermia treatment by emitting body tissue with energy comprising: an applicator; a cooling fluid container holding cooling fluid to be circulated through said applicator; a storage unit for storing said cooling fluid to refill said cooling fluid container; a sensor for detecting whether said cooling fluid's amount held in said cooling fluid container is more than a preset amount or not; a refill control unit refilling said cooling fluid container with said cooling fluid from said storage unit when said cooling fluid's amount in said cooling fluid container is less than the preset amount; a total refill timer counting a total refill time required for refilling said cooling fluid container with said cooling fluid from said storage unit; and an abnormality notifying unit notifying an abnormality when said total refill time exceeds a preset time.

### BACKGROUND OF THE INVENTION

1. Field of the Invention

The present method relates to an apparatus for hyperthermia treatment by emitting an affected region of body tissue with energy such as laser beams, microwaves, radio waves and ultrasonic waves from its emitting unit provided at an insertion part or a pressing part to conduct a hyperthermia treatment after inserting the insertion unit into body cavities or lumens such as blood vessels, urethra, and abdominal cavity, or surgically pressing the pressing part to body tissues body surface.

2. Description of the Related Arts

The apparatus for hyperthermia treatment has been known, wherein a lengthy insertion unit is inserted into a human body through a body cavity or by making a small incision and emitting an affected region with energy such as laser beams, microwaves, radio waves or ultrasonic waves in order to eliminate the lesion area's tissue for treatment through heating, degeneration, necrosis, coagulation, ablation or evaporation. This apparatus for hyperthermia treatment is generally intended to emit directly an affected region in the surface layer of body tissue or its vicinity with energy.

Another technique of emitting deep areas of body tissue with energy for the purpose of treating an affected region located in a deep area of body tissue such as the prostate gland.

This apparatus for hyperthermia treatment is conducted generally in the following sequence, for example, for treating the prostate gland. The operator inserts the insertion unit of the apparatus for hyperthermia treatment into the urethra on his own and causes the emitting unit to reach the urethra in the vicinity of the prostate gland while observing the urethra through the endoscope. Emitting of the energy is conducted by rotating the insertion unit around the axis of the urethra in order to align the emitting unit in the desired direction of emitting.

The energy is emitted from the emitting unit built into the insertion unit. Since the emitting unit emites energy in a very small confined space, it can easily heated to high temperatures. When the emitting unit heats to a high temperature, the emitting unit itself and body tissue adjacent to the emitting unit can be damaged by heat. In order to prevent it, the apparatus for hyperthermia treatment is always cooled by cooling water running close to the emitting unit.

Cooling water is cooled by a cooling device and supplied to the vicinity of the emitting unit by a pump. The cooling device and the insertion unit are connected by a circulation tube and cooling water used for cooling the vicinity of the emitting unit is returned to the cooling device through the circulation tube.

The cooling water is not only circulated for cooling the vicinity of the emitting unit but also for washing the outer surface of the insertion unit and sometimes allowed to flow through the patient's body. Therefore, cooling water should always be maintained clean and fresh. Consequently, a storage tank is prepared for holding fresh cooling water and is connected to the cooling device for each operation.

When cooling water is used for washing the human body, it reduces the amount of cooling water in the cooling device so that cooling water in the cooling device is refilled from the storage tank as needed (see for example Japanese Patent Laid-Open No. 2003-10229).

If cooling water in the apparatus for hyperthermia treatment is used for washing the human body as well, there may be a problem of not having enough cooling water to fill the cooling device as cooling water is consumed for washing of the body too frequently and thus exhausting cooling water held in the storage tank. In such a case, cooling water from the storage tank cannot fill the cooling device even if it is switched to the refilling mode.

The cooling device described above can also have bursting and rupturing accidents in the circulation tube causing leakage of cooling water and thus insufficiency of cooling water in the cooling device.

A problem with such an apparatus for hyperthermia treatment is that it has no way of detecting such an abnormality.

### SUMMARY OF THE INVENTION

The present invention is made to improve such a problem and intends to provide an apparatus for hyperthermia treatment that enable to control a circulation of cooling water as cooling fluid certainly.

The apparatus for hyperthermia treatment by emitting body tissue with energy comprising: an applicator; a cooling fluid container holding cooling fluid to be circulated through said applicator; a storage unit for storing said cooling fluid to refill said cooling fluid container; a sensor for detecting whether said cooling fluid's amount held in said cooling fluid container is more than a preset amount or not; a refill control unit refilling said cooling fluid container with said cooling fluid from said storage unit when said cooling fluid's amount in said cooling fluid container is less than the preset amount; a total refill timer counting a total refill time required for refilling said cooling fluid container with said cooling fluid from said storage unit; and an abnormality notifying unit notifying an abnormality when said total refill time exceeds a preset time.

According to this apparatus for hyperthermia treatment, the operator can easily find out the emptiness of the storage unit or if any loss of fluid has occurred between the storage unit and the cooling fluid container as the operator will be notified of abnormality if the total refill time exceeds the specified time.

Also, the apparatus for hyperthermia treatment by emitting body tissue with energy comprising: an applicator; a cooling fluid container holding cooling fluid to be circulated through said applicator; a storage unit storing said cooling fluid to refill said cooling fluid container; a sensor detecting whether said cooling fluid's amount held in said cooling fluid container is more than a preset amount or not; a refill control unit refilling said cooling fluid container with said cooling fluid from said storage unit when said cooling fluid's amount in said cooling fluid container becomes less than the preset amount; a refill timer counting each refill time required for refilling said cooling fluid container with said cooling fluid from said storage unit; and an abnormality notifying unit notifying abnormality when said refilling time exceeds a preset time.

According to this apparatus for hyperthermia treatment, the operator can easily find out the emptiness of the storage unit or if any loss of fluid has occurred between the storage unit and the cooling fluid container as the operator will be notified of abnormality if the refill time for each refilling cycle exceeds the specified time.

The abnormality notifying method for an apparatus for hyperthermia treatment comprising steps of: refilling a cooling fluid container from a storage unit, wherein said cooling fluid is circulated through an applicator used for emitting body tissue with energy; counting time to refilling said cooling fluid container with cooling fluid as a first counting step; and notifying that there is an abnormality when the time counted by said first counting step exceeds a preset time.

According to the abnormality notifying method of this apparatus for hyperthermia treatment, the operator can easily find out the emptiness of the storage unit or if any loss of fluid has occurred between the storage unit and the cooling fluid container as the operator will be notified of abnormality if the refill time exceeds the specified time.

### BRIEF DESCRIPTION OF THE DRAWINGS

FIG. 1 is a schematic constitution drawing of an apparatus for hyperthermia treatment according to the present invention.

FIG. 2 is a cross-sectional drawing of a delivery tube and a return tube.

FIG. 3 shows a variation of the delivery and return tubes.

FIG. 4 is a diagram showing a control main unit.

FIG. 5 shows a cooling water bag attached to a cooling device.

FIG. 6 is a diagram showing a closing part being opened.

FIG. 7 is a diagram showing a closing part being blocked.

FIG. 8 is a block diagram showing internal constitution of the control main unit.

FIG. 9 is a diagram showing a target operation rate table.

FIG. 10 is a diagram showing a operation rate limitation table.

FIG. 11 is a flow chart showing the operation sequence of the apparatus for hyperthermia treatment in a refilling mode.

FIG. 12 is a flow chart showing the operation sequence of the apparatus for hyperthermia treatment during a interruption process (a).

FIG. 13 is a flow chart showing the operation sequence of the apparatus for hyperthermia treatment in a treatment mode.

FIG. 14 is a flow chart showing the operation sequence of the apparatus for hyperthermia treatment during a interruption process (b).

FIG. 15 is a flow chart showing the operation sequence of the apparatus for hyperthermia treatment during a interruption process (c).

FIG. 16 is a flow chart showing the operation sequence of the apparatus for hyperthermia treatment during a interruption process (d).

FIG. 17 is a diagram showing an example of a cooling water cassette.

FIG. 18 is a diagram showing another example of a cooling water bag.

FIG. 19 is a diagram showing the control main unit.

FIG. 20 is a diagram showing the closing part.

### DETAILED DESCRIPTION OF THE INVENTION

A preferred embodiment of the present invention will be described below with reference to the accompanying drawings.

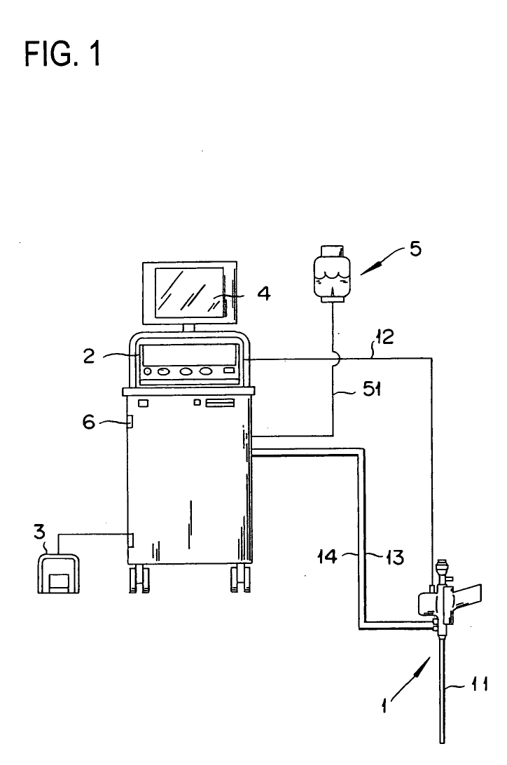

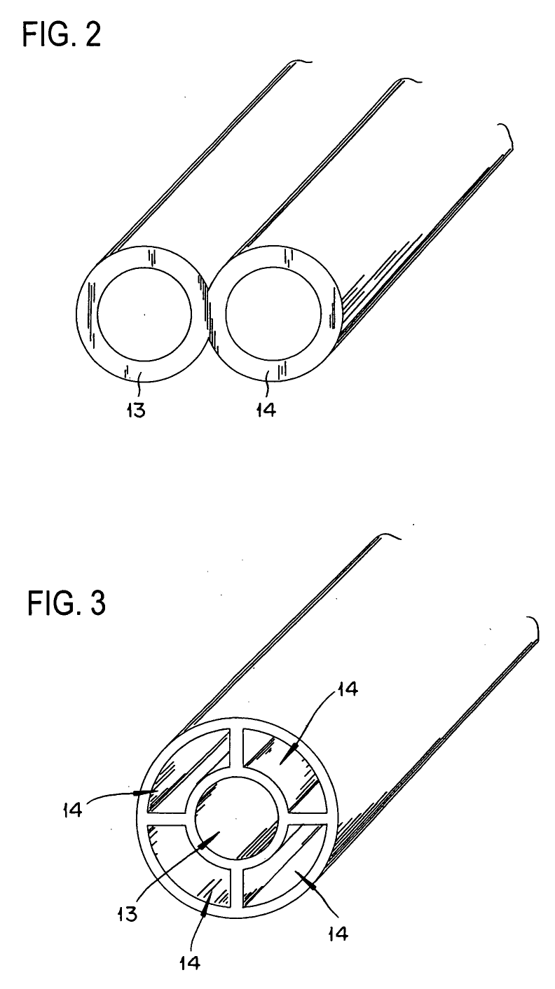

FIG. 1 is a schematic constitution drawing of an apparatus for hyperthermia treatment according to the present invention; FIG. 2 is a cross-sectional drawing of a delivery tube and a return tube; and FIG. 3 shows a variation of the delivery and return tubes.

The apparatus for hyperthermia treatment has an applicator 1, a laser generator 2, a foot switch 3, a display device 4, a storage tank 5 (storage unit), and a control main unit 6. The applicator 1, the laser generator 2, the foot switch 3, the display device 4, and the storage tank 5 are all connected to the control main unit 6 to have their operations controlled. The constitution of each unit will be described below.

The applicator 1 has an elongated insertion unit 11, which is to be inserted into the human body. The insertion unit 11 is provided at its distal end with a laser emitting unit (not shown) for emitting laser beams. The applicator 1 emites body tissue located on its side with laser beams from the laser emitting unit. The radiating laser beams are supplied by the laser generator 2 via an optical fiber 12. The applicator 1 is used to treat benign prostatic hyperplasia and various tumors including cancer by means of laser beam emitting.

In the meanwhile, the laser emitting unit itself is heated because of laser beams and resultantly heats the body surface that is in contact with the laser emitting unit. Overheating of the laser emitting unit also may be a cause of its malfunction and the heating of the surface of normal tissue is preferably be held minimum. Therefore, cooling water as cooling fluid is circulated in the insertion unit 11 so that it will pass through the laser emitting unit.

The applicator 1 is connected to a delivery tube 13 that supplies the cooling water and a return tube 14 that discharges the cooling water so that the cooling water circulates. The cooling water is delivered through the delivery tube 13 to circulate through the applicator 1 and returns through the return tube 14. This enables to cool the laser emitting unit itself located in the insertion unit 11, and also cool the body surface, which is in contact with the insertion unit 11. Both the delivery tube 13 and the return tube 14 are connected to a cooling unit, which is built into the control main unit 6 to be described later. The delivery tube 13 and the return tube 14 are typically formed into an integral piece running parallel to each other as shown in FIG. 2.

The delivery tube 13 and the return tube 14 can be formed concentrically in a single tube as shown in FIG. 3, the delivery tube 13 being located inside and the return tube 14 being located outside.

The applicator 1 further has a flush lumen (not shown). The flush lumen is a passage formed branching from the cooling water circulation route in the insertion unit 11 and extending to the tip of the insertion unit 11. The flush lumen is normally closeed. In an event of washing a laser emitting window located in the vicinity of the distal end of the insertion unit 11 and an endoscopic observation window, the closing is released and the cooling water is ejected to the outside from the insertion unit 11. Since the cooling water is used for washing, the cooling water within the control main unit 6 reduces due to the flushing operation.

The laser generator 2 generates laser beams used for emitting the body. The laser generator 2 is used for setting up output conditions such as laser power value, laser pulse time, laser pulse interval through switches and dials.

The laser generator 2 is connected to the foot switch 3 via the control main unit 6. When the operator is pressing down the foot switch 3, the power to the laser generator 2 is kept ON to generate laser beams. This enables the operator to emit the lesion with laser beams for a arbitrary timing and duration.

The foot switch 3 provided at the operator's foot generates ON and OFF signals to prompt the control main unit 6 to cause laser beam emitting. When the laser emitting preparation procedure is completed and the foot switch 3 is stepped on, the laser generator 2 radiates laser beams.

The display device 4 located on top of the control main unit 6. The display device 4 is a user interface for displaying specific information for the operator as well as for accepting various setups and operational instructions. The display device 4 is made as a touch panel system so that the operator can control the system by simply touching the screen surface.

The storage tank 5 stocks cooling water and refines the cooling device built into the control main unit 6 with cooling water. The cooling water to be stocked can be sterilized water, sterilized distilled water, or sterilized physiological saline. The cooling water is supplied from the storage tank 5 to the control main unit 6 via a refilling tube 51. The storage tank 5 is plugged by a rubber cap at the bottom. The distal end of the refilling tube 51 is formed in a needle shape. In order to connect the storage tank 5 with the control main unit 6, the needle like end of the refilling tube 51 is inserted into the bottom of the storage tank 5. This construction simplifies the connection and replacement of the storage tank 5 while maintaining a good sealing characteristic.

The storage tank 5 is preferably made as a soft bag deformable by the atmospheric pressure when its internal pressure becomes negative. If the storage tank 5 is built as a hard case, it is preferable to be provided with a breathing hole.

The control main unit 6 is connected with various units of the apparatus for hyperthermia treatment as described above, and controls the operation of the entire apparatus for hyperthermia treatment based on ON/OFF signals from the foot switch 3 and others. The control main unit 6 also controls the cooling water refilling of the storage tank 5. In particular, the present invention is characterized by the constitution that the control main unit 6 detects various conditions of abnormality such as lack and leakage of the cooling water in refilling with the cooling water from the storage tank 5.

Therefore, the constitution of the control main unit 6 will be described below.

(Control Main Unit)

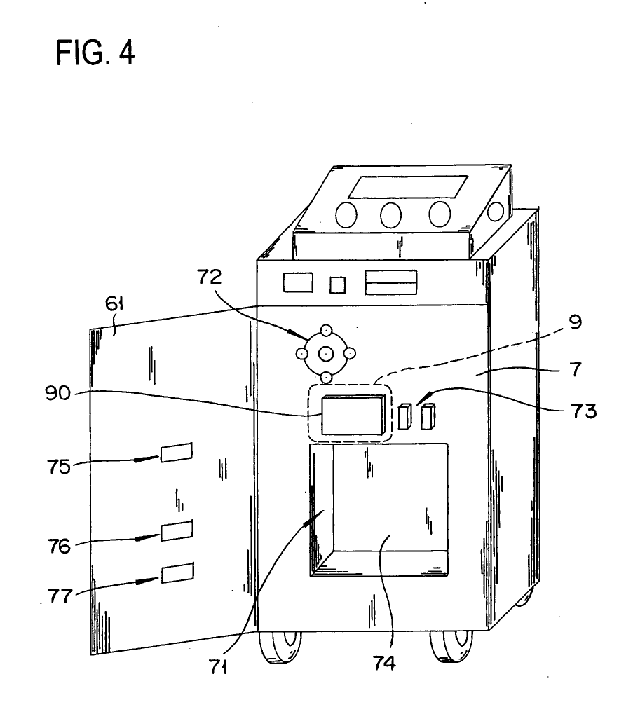

FIG. 4 is a diagram showing the control main unit.

The control main unit 6 has a door 61 and has a cooling device 7 inside the control main unit enclosed by the door 61.

The door 61 has a door opening sensor (not shown). When the door 61 is open, the cooling water refilling cannot be done.

The cooling device 7 is equipped with a storage 71, a rotary pump 72, a flow sensor 73, a temperature adjusting unit 74, an upper limit sensor 75, a lower limit sensor 76, a temperature sensor 77, and such a closing part 9 as a pinch valve.

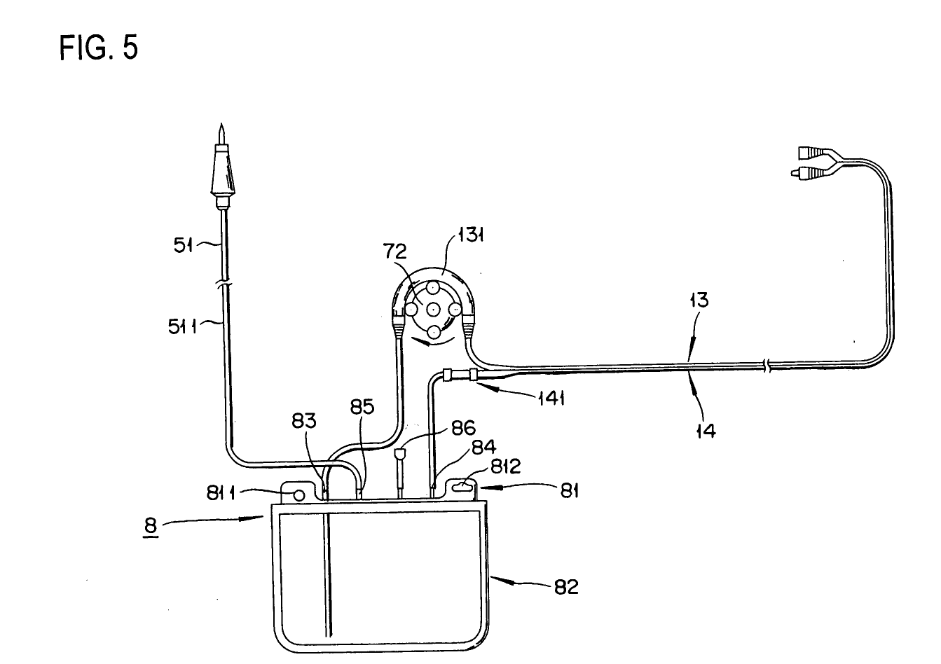

The storage 71 is a cavity in the control main unit 6 providing a specified internal space when the door 61 is closed. The storage 71 stores a cooling water bag 8 (see FIG. 5) which serves as a container of the cooling water. The cooling water bag 8 is positioned and mounted using a mounting pin (not shown) provided in the storage 71. The aforementioned the delivery tube 13 and the return tube 14 extend from the cooling water bag 8. The cooling water bag 8 will be described in detail later with reference to FIG. 5.

The rotary pump 72 is provided on top of the storage 71. The rotary pump 72 is attached rotatably and is equipped with spherical members on its outer periphery. As the rotary pump 72 rotates, it squeezes a portion of the delivery tube 13 extending from the cooling water bag 8 with its spherical member to provide a pressure to cause the cooling water in the tube to be pressured to circulate through the applicator 1.

The flow sensor 73 is, for example, a proximity sensor (photodiode, etc.) It detects the flow of the cooling water by the rotation of a metal waterwheel placed in the return tube 14.

The temperature adjusting unit 74 is provided in the storage 71. When the cooling water bag 8 is placed in the storage 71 and it is filled with cooling water, the temperature adjusting unit 74 makes contact with the cooling water bag 8 to heat or cool the cooling water bag 8 thus to control the temperature of the cooling water.

The temperature adjusting unit 74 consists of a Peltier device. A Peltier device is a device that becomes cooler when a DC current runs in a specific direction and becomes hotter when a DC current runs in the opposite direction. It is also possible to change the Peltier device's cooling or heating power by changing the electric power.

The upper limit sensor 75 is provided on the door 61 and detects as to whether the cooling water volume in the cooling water bag 8 is above the specified upper limit or not. The lower limit sensor 76 detects whether the cooling water volume in the cooling water bag 8 is below the specified lower limit or not. The upper limit sensor 75 and the lower limit sensor 76 are both capacitance type proximity sensors. They can also be a type of optically detecting the water level or a type that directly detecting the water level in the cooling water bag 8.

The temperature sensor 77 is provided on the door 61 and detects the cooling water's temperature by means of contacting the cooling water bag 8.

The closing part 9 closes and opens the aforementioned refilling tube 51 connecting with the storage tank 5 and the cooling water bag 8. This controls the cooling water refillment from the storage tank 5 to the cooling water bag 8.

The cooling water bag 8 and the closing part 9 are described in more detail below.

(Cooling Water Bag)

FIG. 5 shows a cooling water bag attached to a cooling device.

The cooling bag 8 is equipped with a hanger 81 and a bag 82 as shown in FIG. 5.

The hanger 81 is made of harder material such as 5 mm thick polycarbonate and the bag 82 is made of a softer material such as 100 μm PET.

The hunger 81 is provided with a hole 811 and a hole 812 so that the cooling bag 8 can be hung on mounting pins (not shown) in the storage 71 in installing it in the storage 71. The hole 811 and the hole 812 are shaped differently and so are their mating pins of the storage 71. Therefore, the cooling water bag 8 is mounted on the storage 71 in only one direction. This prevents the delivery tube 13 and the return tube 14 extending from the cooling bag 8 from being installed in the applicator 1 in the opposite direction.

The hunger 81 is provided with a delivery tube port 83, a return tube port 84, a refilling tube port 85, and an air vent 86.

The delivery tube port 83 is connected to the delivery tube 13. The delivery tube port 83 is for sucking cooling water in the cooling water bag 8 so that it extends deeply inside the bag 82. The delivery tube 13 is provided with a pump tube 131. The pump tube 131 contacts with the rotary pump 72 when the cooling water bag 8 is attached to the storage 71. As the rotary pump 72 is rotated in the clockwise direction as shown in FIG. 5, the pump tube 131 is squeezed by it providing a pressure to the cooling water inside the pump tuber 131 to circulate toward the applicator 1.

The return tube port 84 is connected to the return tube 14. The return tube 14 is provided with a waterwheel unit 141. The waterwheel unit 141 contains a metal waterwheel inside. This waterwheel's rotary speed is detected by the flow sensor 73 shown in FIG. 4. The flow sensor 73 detects the rotary speed and hence the flow volume of the cooling water flowing through said return tube 14.

The refilling tube port 85 is connected to the refilling tube 51. The refilling tube 51 is held at a position 511 by the closing part to be described later in order to be closed or opened. The air vent 86 is a filter for preventing the cooling water in the cooling water bag 8 from passing through, while allowing only air to pass through.

The bag 82 is formed to be hollow in the inside by gluing two flexible sheets together. The bag 82 is glued below the hunger 81. Therefore, if the hunger 81 is mounted in the inside of the storage 71, the bag 82 is automatically positioned in the specified position. The specified position means the position where the broad surface of the bag 82 is automatically makes a close contact with the temperature adjusting unit 74 and contacts with the upper limit sensor 75 provided on the inside of the door 61, the lower limit sensor 76, and the temperature sensor 77 as well, when the cooling water bag 8 is filled with the cooling water and the door 61 is closed.

The bag 82 is formed of a flexible material as mentioned before. Therefore, it is deflated before it is filled with the cooling water but inflates as it is filled with the cooling water and makes a close contact with the temperature adjusting unit 74 and others when it is full.

(Closing Part)

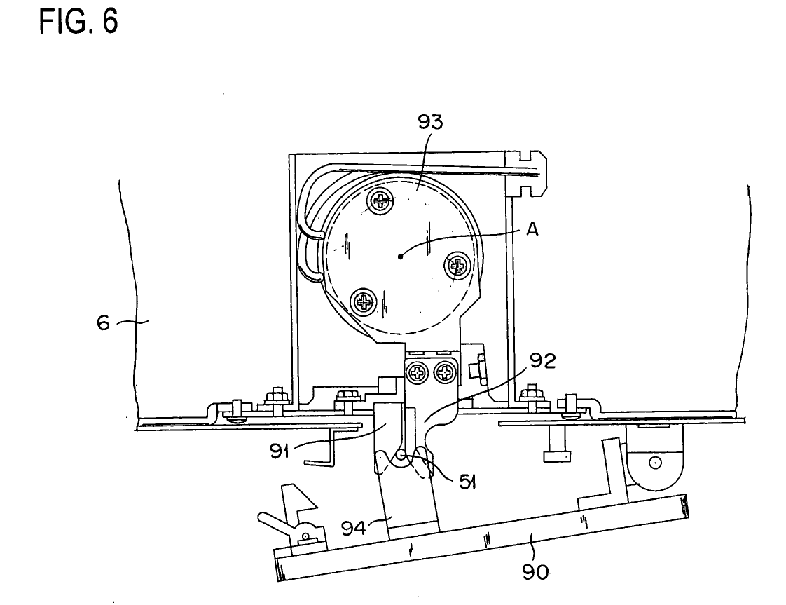

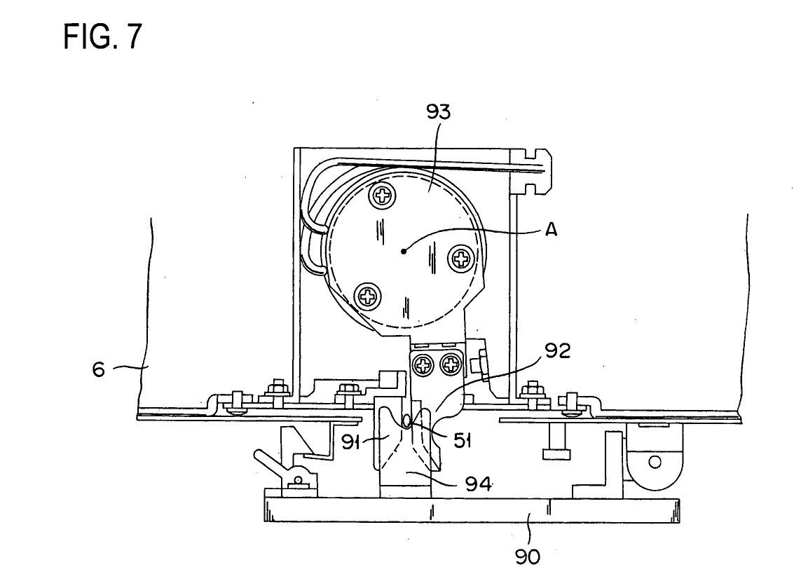

FIG. 6 shows the closing part when it is open, and FIG. 7 shows it when it is blocking. Both FIG. 6 and FIG. 7 show plan views of the closing part 9. Each of them shows a cross section of the refilling tube 51.

The closing part 9 is equipped with a small door 90, a fixed piece 91, a movable piece 92, a rotary solenoid 93, and a holding piece 94.

The small door 90 is mounted on the control main unit 6. Opening the small door 90 reveals that the control main unit 6 has a notch allowing the fixed piece 91 and the movable piece 92 to stick out.

Both the fixed piece 91 and the movable piece 92 are formed in such a way that their widths narrow toward each other. As shown in FIG. 6, when the movable piece 92 has not moved yet, the gap between the fixed piece 91 and the movable piece 92 is substantially V-shaped. When the cooling water bag 8 is mounted on the cooling device 7, the refilling tube 51 is located in the middle of the fixed piece 91 and the movable piece 92, i.e., in the bottom of the V-shape.

The movable piece 92 is attached to the rotary solenoid 93 so that it is always energized by the elastic force of the rotary solenoid 93 in the direction of abutting the fixed piece 91 (clockwise in FIG. 6).

The rotary solenoid 93 is rotatable around the axis A using attractive and repulsive forces between a built-in coil and a permanent magnet as the current runs.

The holding piece 94 is mounted on the inside of the small door 90 and its point is formed in a smooth concave shape. The holding piece 94 catches the refilling tube 51 with the concave shape of its tip when the small door 90 is closed, thus causing the refilling tube 51 to be pressured against the fixed piece 91 and the movable piece 92. The refilling tube 51, as it is pressured, enters the gap between the fixed piece 91 and the movable piece 92. Here, the movable piece 92 rotates counterclockwise in accordance with the movement of the refilling tube 51 resisting the elastic force of the rotary solenoid 93 around the axis A as shown in FIG. 7.

When the small door 90 is completely closed, the refilling tube 51 is pinched between the fixed piece 91 and the movable piece 92 and is energized by the elastic force of the rotary solenoid 93, so that it becomes squeezed into an elliptical cross section as shown in FIG. 7 and the passage is blocked.

In order to relieve the refilled tube 51 from the condition closed by the closing part 9, the rotary solenoid 93 is electrically energized. The electrical energized rotary solenoid 93 rotates in the direction opposite to the elastic force so that the movable piece 92 moves in the direction of parting away from the fixed piece 91. This cause the gap between the fixed piece 91 and the movable piece 92 to widen, thus allowing the refill tube 51 to restore its original cross section shape. Therefore, the refilling tube 51 allows the cooling water to pass through.

Thus, electrical energization of the rotary solenoid 93 uncloses the refilling tube 51, and stopping the electrical energization blocks it. The refilling tube 51 is unclosed when refilling the cooling water from the storage tank 5 to the cooling water bag 8, and is closed when canceling the refilling.

In comparing the fixed piece 91 and the movable piece 92, the opposing straight line portion of the fixed piece 91 is longer than that of the fixed piece 92. Therefore, when the refilling tube 51 is first being pushed into the gap between the fixed piece 91 and the movable piece 92, it makes a contact with the straight line portion of the movable piece 92 before it makes a contact with the straight line portion of the fixed piece 91. This makes the pushing force of the refilling tube 51 acts to move the movable piece 92.

Next, the internal constitution of the control main unit 6 is described with reference to a block diagram.

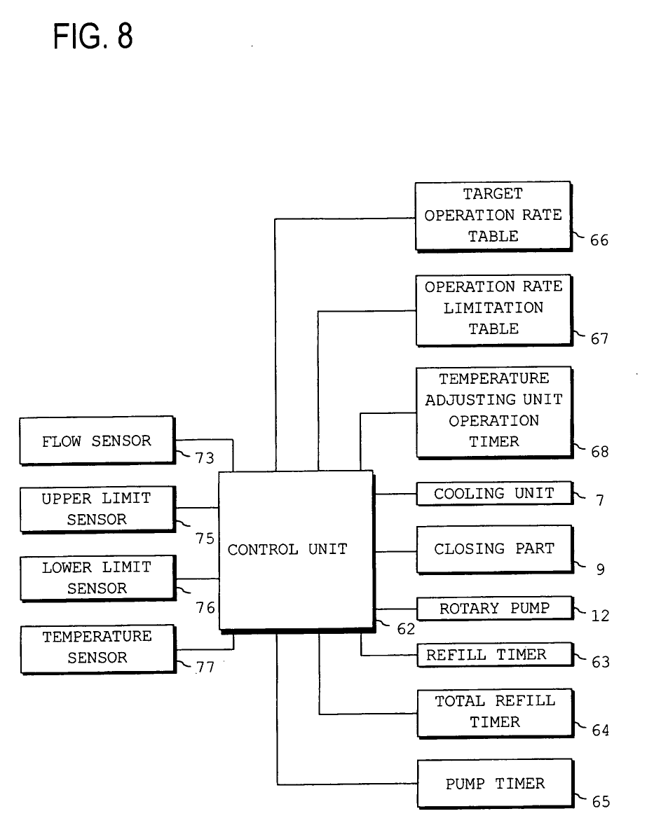

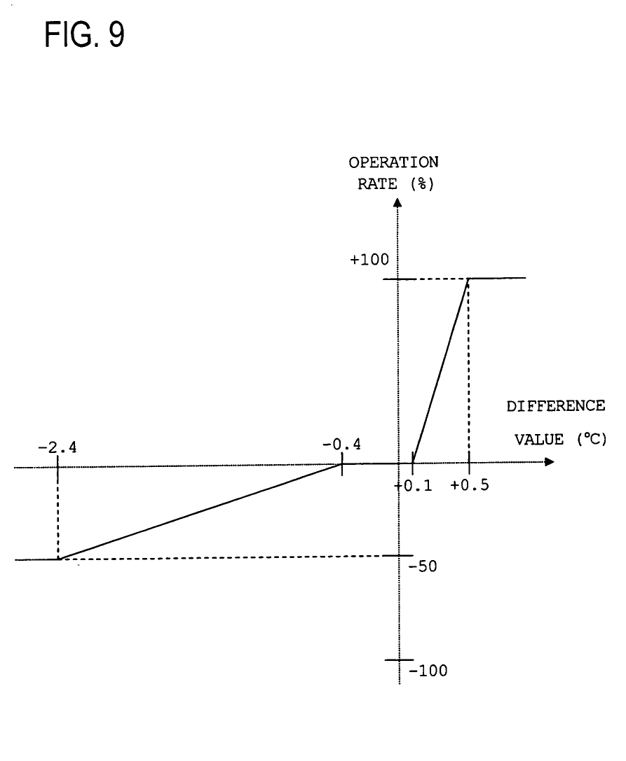

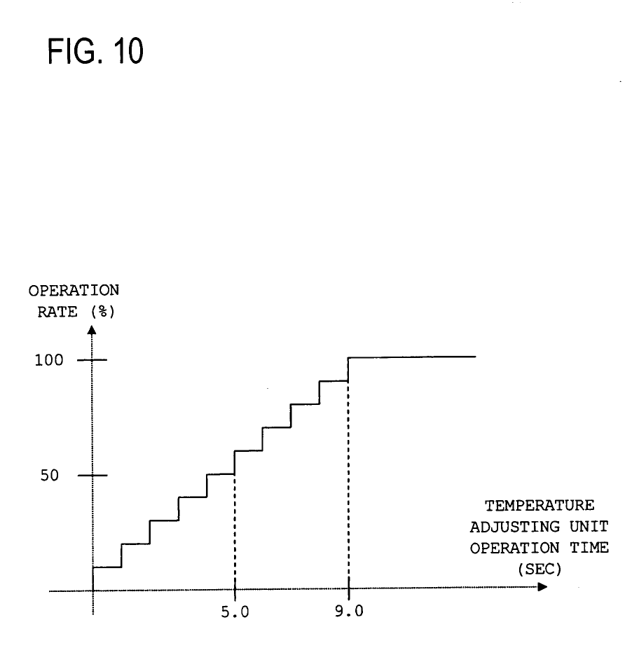

FIG. 8 is a block diagram showing the internal constitution of the control main unit 6; FIG. 9 is a target operation rate table; and FIG. 10 shows a operation rate limitation table.

As shown in FIG. 8, the control main unit 6 contains, in addition to the abovementioned components, a control unit 62, a refill timer 63, a total refill timer 64, a pump timer 65, a target operation rate table 66, an operation rate limitation table 67, and a temperature adjusting unit operation timer 68. The refill timer 63, the total refill timer 64, the pump timer 65, the target operation rate table 66, the operation rate limitation table 67, and the temperature adjustment unit operation timer 68 are all connected to the control unit 62.

The control unit 62 is connected with various components of the apparatus for hyperthermia treatment and generally controls the actions of the entire apparatus for hyperthermia treatment. The control unit 62 receives the signals from the upper limit sensor 75, the lower limit sensor 76, and the temperature sensor 77 to control the cooling device 7 and the closing part 9.

The refill timer 63 measures via the control unit 62 the time period the closing part 9 is open, in other words, the time period the cooling water is being refilled from the storage tank 5 to the cooling water bag 8.

The total refill timer 64 measures via the control unit 62 the total time period when the closing part 9 is open throughout a single treatment.

The pump timer 65 measures via the control unit 62 the time period since the rotary pump 72 has started to run.

The target operation rate table 66 shown in FIG. 9 is a table for showing the relation between the difference value between the measured temperature and the target temperature (specified preset temperature) and the power intensity applied on the temperature adjusting unit 74. In FIG. 9, the difference value shown on the horizontal axis is a value obtained by subtracting the specified preset temperature from the temperature of the cooling water measured by the temperature sensor 77. The operation rate shown on the vertical axis is a value at which percentage of the maximum power the temperature adjusting unit 74 should be operated. If the operation rate is +100%, it means that the temperature adjusting unit 74 be operated to cool by 100% of the maximum power; if the power intensity is −50%, the temperature adjusting unit 74 is operated to heat at the 50% of its capacity. Whether the system is to be warmed or cooled is controlled by the direction of the current, and the percentage represents the intensity of the current used to operate the system.

According to FIG. 9, it is set in such a way that cooling occurs when the difference value becomes higher than +0.1° C., 100% operation cooling occurs when it is higher than +0.5° C., heating starts when it becomes lower than −0.4° C., and 50% heating occurs when it is lower than −2.4° C. in the present embodiment.

The operation rate limiting table 67 shown in FIG. 10 is a table showing the relation between the measured value of the temperature adjusting unit operation timer 68 and the maximum operation rate of the temperature adjusting unit 74. In FIG. 10, the measured value shown on the horizontal axis is the time elapsed (seconds) since the temperature adjusting unit 74 started to operate. The maximum operation rate shown on the vertical axis is a value at which percentage of the maximum power the temperature adjusting unit 74 should be operated.

The temperature adjusting unit operation timer 68 measures the time elapsed since the temperature adjusting unit 74 started to operate.

Let us now describe how the target operation rate table 66, the operation rate limitation table 67, and measurement data from the temperature adjusting unit operation timer 68 are referenced for conducting the temperature adjustment control.

For example, if the preset temperature of the cooling water is 22° C. and the current measured temperature of the cooling water is 23° C., it is learned that the difference value between the preset temperature and the current temperature is +1° C. by referencing the target operation rate table 66 shown in FIG. 9. Therefore, the target operation rate is +100%, i.e., it is decided that it is to be cooled at 100% operation rate.

Furthermore, the operation rate limitation table 67 shown in FIG. 10 is referenced here based on the measured time of the temperature adjusting unit operation timer 68. In other words, instead of operating the temperature adjusting unit 74 at a operation rate of 100%, the operation rate is increased gradually based on the measured time of the temperature adjusting unit operation timer 68. The operation rate is set to 50% after 5 seconds from the start of operating and 100% after 9 seconds. This prevents excessively sharp cooling of the cooling water.

As to the target operation rate table 66 shown in FIG. 9, the operation rate of the temperature adjusting unit 74 shall be maintained at 50% once it is set to 50% and the operation time has elapsed 5 seconds. When the cooling water temperature reaches the preset temperature, the operation of the temperature adjusting unit 74 is stopped.

When the temperature adjusting unit 74 stops, the temperature adjusting unit operation timer 68 is counted down and the operation rate of the temperature adjusting unit 74 starts to be gradually reduced until it reaches 0% and stops.

Cooling and heating by the temperature adjusting unit 74 is adjusted in steps like this prevent sharp temperature changes and degeneration of the Peltier device used in the temperature adjusting unit 74.

(Action)

The above has been a description of the constitution of the apparatus for hyperthermia treatment according to the present invention. Now, we will describe how the apparatus for hyperthermia treatment constituted as such operates taking an example of applying the apparatus for hyperthermia treatment for treating prostatic disease. We assume that the cooling water bag 8 is already installed in the cooling device 7.

As the refilling mode prior to applying the apparatus for hyperthermia treatment to the treatment, the cooling water is refilled from the storage tank 5 to the cooling water bag 8 by connecting the storage tank 5 to the refilling tube 51, and then the applicator 1 is inserted into the patient's body to start treatment in the treatment mode. The operation of the apparatus for hyperthermia treatment is described below in two stages, i.e., the refilling mode and the treatment mode. The entire operation of the apparatus for hyperthermia treatment is controlled by said control unit 62.

<Refilling Mode>

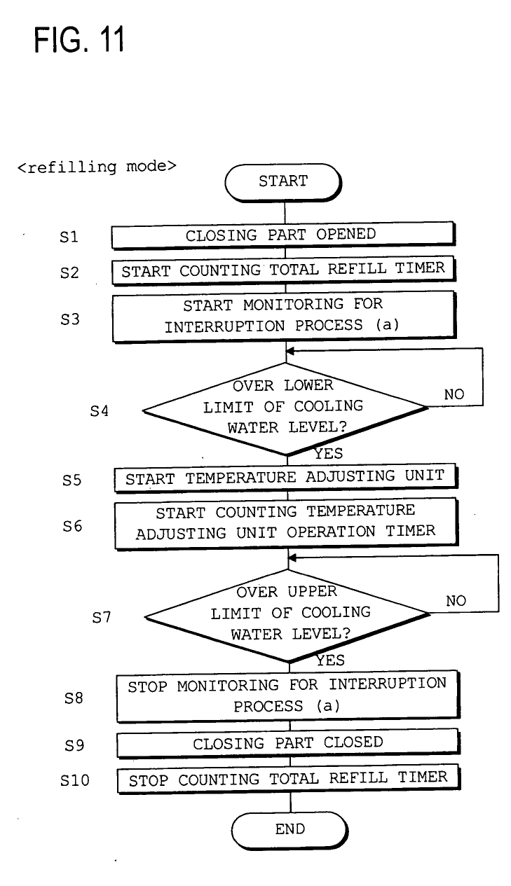

FIG. 11 is a flow chart for showing the operation sequence of the apparatus for hyperthermia treatment in a refilling mode.

In the refilling mode, the cooling water for cooling the urethra surface during laser emitting in the cooling water bag 8 of the cooling device 7 is refilled.

First, the closing at the location 511 of the refilling tube 51 is relieved in the closing part 9 (step S1). This makes it possible for the cooling water to pass through the refilling tube 51 so that the cooling water is fed by gravity from the storage tank 5, which is located higher than the cooling bag 8, to the cooling water bag 8.

The measurement by the total filling timer 64 starts at this point (step S2). At the same time, monitoring for judging as to whether the interruption process (a) is required or not (step S3). This monitoring is done by the control unit 62. How to advance to the interruption process (a) will be described later.

A judgment will be made as to whether the cooling water bag 8 is refilled with the cooling water to exceed the lower limit or not based on the detection of the lower limit water sensor 76 (step S4). If the cooling water has not been refilled to exceed the lower limit (step S4: No), the detection of the lower limit sensor 76 will be continued.

When the cooling water level has reached the lower limit (step S4: Yes), the temperature adjusting unit 74 starts to be operated (step S5), and the temperature adjusting unit timer 68 starts to count simultaneously (S6). At this point, the temperature of the cooling water is adjusted while referencing the target operation rate table 66 of FIG. 9, the operation rate limitation table 67 of FIG. 10, and the measured time of the temperature adjusting unit operation timer 68 as mentioned before.

A judgment is made as to whether the cooling water level has reached the upper limit of the cooling water bag 8, i.e., where the cooling water bag 8 is fully refilled or not based on the detection of the upper limit water sensor 75 (step S7). If the bag is not refilled fully with the cooling water (step S7: No), the cooling water refilling process will be continued.

If the bag is refilled fully with the cooling water (step S7: Yes), the monitoring for the interruption process (a) is terminated (step S8), the refilling tube 51 is closed by the closing part 9 (step S9). This terminates the refilling of the cooling water bag 8 with the cooling water. Simultaneously, the measurement by the total refill timer 64 is temporarily stopped (step S10). The measurement result of the total refill timer 64 is maintained.

Interruption Process (a)

We will now describe the interruption process (a), which can be executed during the steps S3 through step S8 of the refilling mode described above.

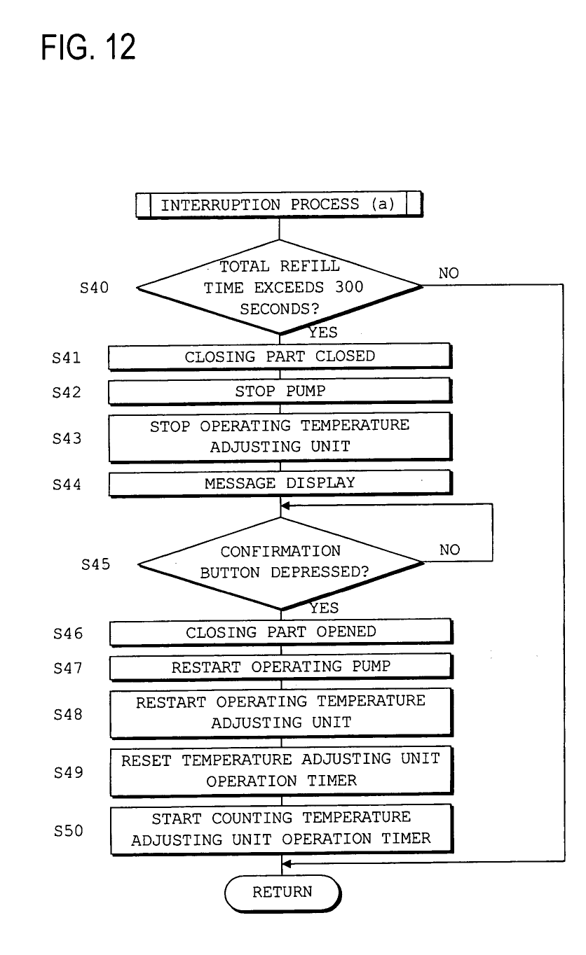

FIG. 12 is a flow chart showing the operation sequence of the apparatus for hyperthermia treatment during the interruption process (a).

A judgment is made as to whether the measurement time by the total refill timer 64 has passed a preset time (e.g., 300 seconds) or not (step S40), and it essentially advances to the interruption process (a) if it has exceeded 300 seconds (step S40: Yes). If it has not passed 300 seconds (step S40: No), the interruption process (a) is not executed and the refilling mode is continued.

When it advances to the interruption process (a), the refilling tube 51 will be closed by the closing part 9 (step S41). When the rotary pump 72 is operating, the rotary pump 72 is stopped (step S42), and the temperature adjusting unit 74 is stopped as well (step S43).

Then, a message is displayed on the display device 4 (step S44). The displayed message can be, for example, “Check the remaining amount in the storage tank.” “Check if the refilling tube has slipped off or damaged,” “Check if the cooling water bag is damaged,” etc.

The control unit 62 makes a judgment whether a confirmation button displayed on the display device 4 is pressed or not (step S45), and waits until the confirmation button is pressed. When the confirmation button is pressed (step S45: Yes), the closing part 9 is released so that the system returns to the refilling mode (step S46). If necessary, the operation of the rotary pump 72 is restarted (step S47). The operation of the temperature adjusting unit 74 is also restarted (step S48).

Then, the temperature adjusting unit operation timer 68 is reset (step S49), and the measurement restarts (step S50).

When the system advances from the refilling mode into the interruption process (a), the rotary pump 72 normally has not started to run. However, since there is a case when it advances to the interruption process (a) from the interruption process (b), the step S42 and the step S47 are provided for operating the rotary pump 72.

As can be seen from the above, the total refill timer 64 monitors if the set time (e.g., 300 seconds) has been exceeded during the refilling of the cooling water bag 8 with cooling water in the refilling mode, and issues a warning message when it detects that the set time is exceeded judging that it is abnormal. Therefore, it is capable of informing the operator that there is a possibility of slip off or damage of the refilling tube 51 or the cooling water bag 8.

<Treatment Mode>

Next, the treatment mode will be described.

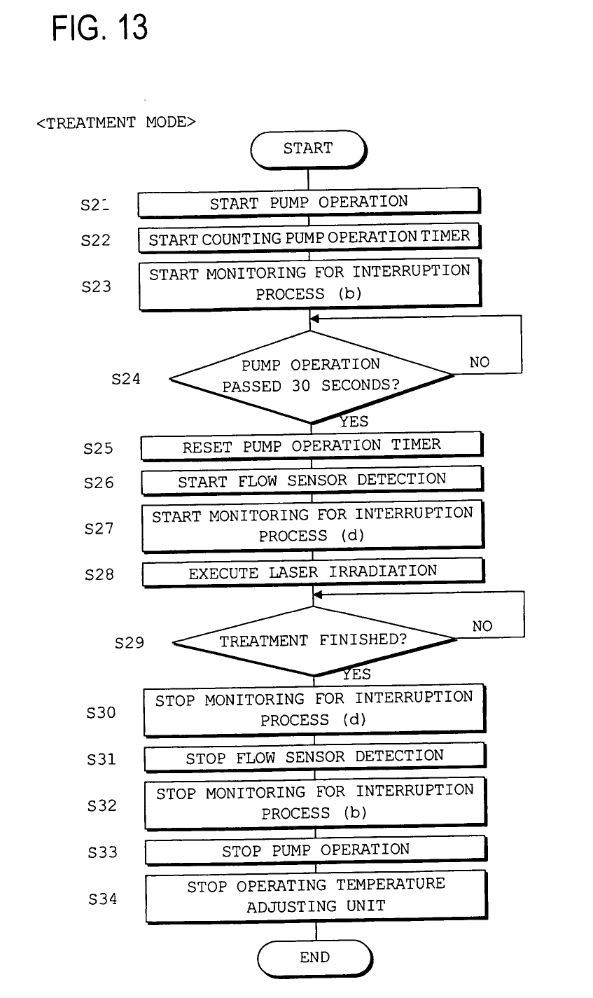

FIG. 13 is a flow chart showing the operation sequence of the apparatus for hyperthermia treatment in a treatment mode.

After the refilling mode is finished, then comes the treatment mode.

First, the operator instructs the system to start the rotary pump 72 (step S21), the measurement of the time by the pump timer 65 is initiated (step S22). Monitoring for judging whether to advance to the interruption process (b) or not is initiated simultaneously (step S23). A case of advancing to the interruption process (b) will be described later.

A judgment is made as to whether the time measured by the pump timer 65 has passed 30 seconds or not (step S24). If the time measured by the pump timer 65 has not passed 30 seconds (step S24: No), it waits until it passes.

If it has passed 30 seconds (step S24: Yes), the pump timer 65 is reset (step S25), and detection of the cooling water's flow volume by the flow sensor 73 starts (step S26). Next, monitoring for judging whether to advance to the interruption process (d) or not is initiated (step S27).

When the operator steps on the foot switch 3, laser beam emitting starts (step S28). Laser beam emitting is performed continuously or intermittently until the treatment is finished. When the treatment is finished (step S29: Yes), monitoring for judging whether to advance to the interruption process (d) or not is terminated (step S30), and detection by the flow sensor 73 is terminated as well (step S31).

Furthermore, monitoring for judging whether to advance to the interruption process (b) or not is terminated (step S32), the rotary pump 72 is stopped (step S33), and the temperature adjusting unit 74 stops to operate (step S34).

We will now describe the interrupting process (b), which can be executed during the steps S23 through step S32 of the treatment mode described above.

Interrupting Process (b)

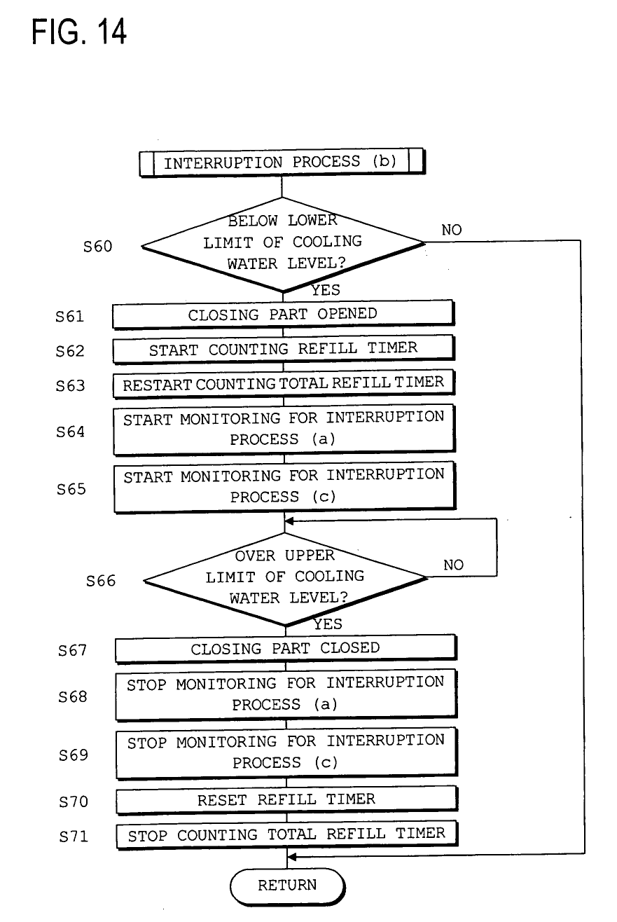

FIG. 14 is a flow chart showing the operation sequence of the apparatus for hyperthermia treatment during a interruption process (b).

A judgment is made whether the cooling water level of the cooling water bag 8 is below the upper limit or not based on the detection of the upper limit sensor 75 (step S60) and it advances essentially to the interruption process (b) if it is below the upper limit (step S60: Yes). If the cooling water level is not below the upper limit (step S60: No), the interruption process (b) is not executed and the treatment mode is continued.

When it advances to the interruption process (b), the refilling tube 51 will be opened by the closing part 9 (step S61). This starts the refilling of the cooling water bag 8 with the cooling water. At the same time, the refill timer 63 starts its counting from zero (step S62), and the total refill timer 64 starts its counting as well (step S63). Furthermore, it starts monitoring for a judgment of whether it should advance to the interruption process (a) or to the interruption process (c) (steps S64 and step S65).

It makes a judgment whether the cooling water level exceeds the upper limit or not as a result of the cooling water refilling (step S66) and waits until it reaches the upper limit. When it exceeds the upper limit (step S66: Yes), it determines that enough cooling water is supplied so that the closing part 9 closes the refilling tube 51 (step S67).

This terminates the monitoring for advancing to the interruption process (a) and interruption process (c) (steps S68 and S69). When the refill timer 63 is reset (step S70), the counting of the total refill timer 64 stops temporarily (step S71).

As can be seen from the above, the present invention allows the cooling water to be refilled automatically when it detects a lack of sufficient amount of the cooling water by means of the interruption process (b) even during the treatment mode.

Furthermore, there are circumstances that it advances to the interruption process (a) and the interruption process (c) even during said interruption process (b). The interruption process (a) is executed before the steps S64 through S68 in said interruption process (b). Since the condition for advancing to the interruption process (a) and the contents of the process have been described already, their descriptions are not repeated here. The interruption process (c) will be described below.

Interrupting Process (c)

The interruption process (c) can be executed before the steps S65 through S69 in said interruption process (b).

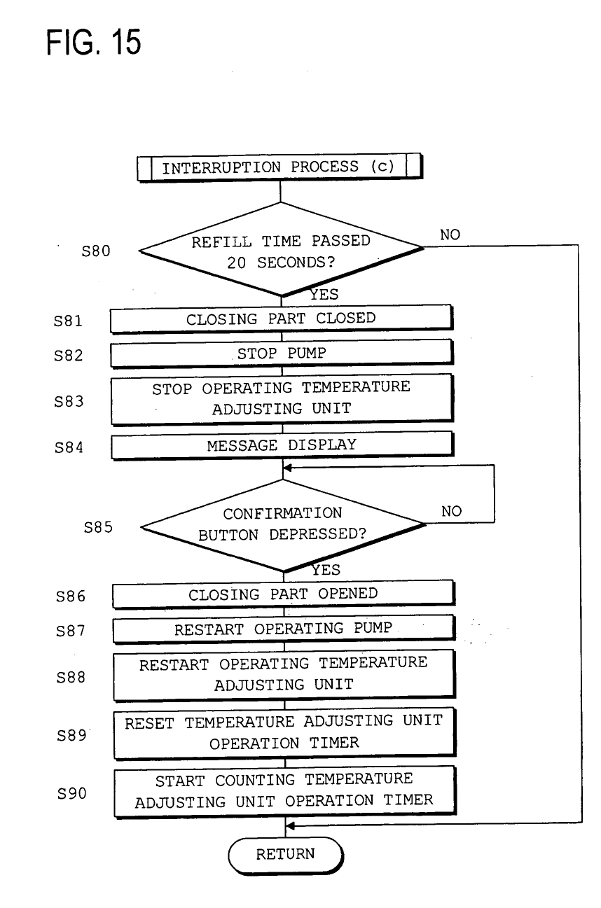

FIG. 15 is a flow chart showing the operation sequence of the apparatus for hyperthermia treatment during a interruption process (c).

A judgment is made as to whether the time measured by the filling timer 63 since it was started in the step S62 of the interruption process (b) has passed 20 seconds (step S80), and it essentially advances to the interruption process (c) if it has passed more than 20 seconds (step S80: Yes). If it has not passed 20 seconds (step S80: No), the interruption process (b) will be continued.

When it advances to the interruption process (c), the refilling tube 51 will be closed by the closing part 9 (step S81). This terminates the refilling of the cooling water bag 8 with the cooling water. The operation of the rotary pump 72 is stopped (step S82), and the operation of the temperature adjusting unit 74 is also stopped (step S83).

Then, a message is displayed on the display device 4 (step S84). The displayed message can be, for example, “Check if the refilling tube has slipped off or damaged,” “Check if the cooling water bag is damaged,” etc.

The control unit 62 makes a judgment whether a confirmation button displayed on the display device 4 is pressed or not (step S85), and waits until the confirmation button is pressed. When the confirmation button is pressed (step S85: Yes), the closing part 9 is released so that the system returns to the interruption process (b) (step S86). The operation of the rotary pump 72 is restarted (step S87). The operation of the temperature adjusting unit 74 is also restarted (step S88).

Then, the temperature adjusting unit operation timer 68 is reset (step S89), and the measurement restarts (step S90).

As can be seen from the above, the refill timer 63 measures the time for refilling the cooling water bag 8 with the cooling water and displays a warning message on the display device 4 judging that it is abnormal if it is longer than the set time (e.g., 20 seconds) in the interruption process (b). Therefore, it is capable of informing the operator that there is a possibility of slip off or damage of the refilling tube 51 or the cooling water bag 8.

We will now describe the interrupting process (c), which can be executed during the steps S27 through step S30 of the treatment mode described above.

Interrupting Process (d)

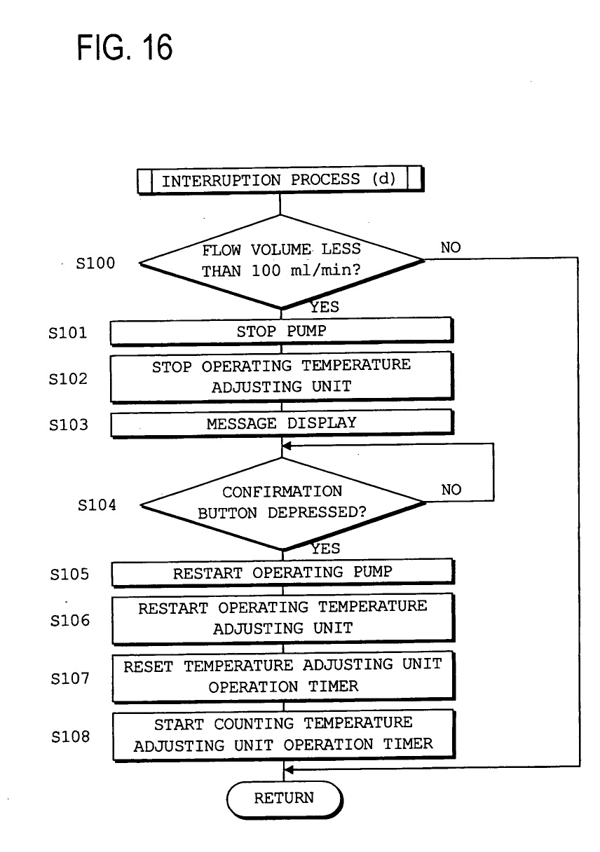

FIG. 16 is a flow chart showing the operation sequence of the apparatus for hyperthermia treatment during a interruption process (d).

A judgment is made as to whether the flow volume of the cooling water circulating between the applicator 1 and the cooling water bag 8 is below a set flow volume (e.g., 100 ml/min) based on the measurement by the flow sensor 73 started in the step S26 of the treatment mode (step S100), and it essentially advances to the interruption process (d) if it is below the set flow volume (step S100: Yes). If it is above the set flow volume (step S100: No), the treatment mode is continued.

The operation of the rotary pump 72 is stopped (step S101) if it advances to the interruption process (d), and the operation of the temperature adjusting unit 74 is also stopped (step S102).

Then, a message is displayed on the display device 4 (step S103). The displayed message can be, for example, “Check if there is any water leakage in the delivery tube or the return tube,” “Check there is any bending or clogging in the delivery tube or the return tube,” etc.

The control unit 62 makes a judgment whether a confirmation button displayed on the display device 4 is pressed or not (step S104), and waits until the confirmation button is pressed. When the confirmation button is pressed (step S104: Yes), the rotary pump 72 is restarted so that the system returns to the treatment mode (step S105). The operation of the temperature adjusting unit 74 is also restarted (step S106).

Then, the temperature adjusting unit operation timer 68 is reset (step S107), and the measurement restarts (step S108).

As can be seen from the above, the flow sensor 73 monitors the flow volume of the circulating cooling water and determines that abnormality exists when it is below the set flow volume and displays a warning message on the display device 4 in the interruption process (d). Therefore, it is capable of detecting abnormal conditions such as leakage, bending and clogging of the delivery tube 13 and the return tube 14 and notifying the operator.

In the treatment mode, the flow sensor 73 detects the flow volume from 30 seconds after the rotary pump 72 starts to run. This is because the rotary pump 72 is provided in the delivery tube 13 and the flow sensor 73 is provided in the return tube 14. In other words, the cooling water does not reach the flow sensor 73 of the return tube 14 immediately after the start of the rotary pump 72, i.e., there is a time lag before the flow reaches there.

By delaying the detection timing of the flow sensor 73 by the time lag (e.g., 30 seconds), it is possible to prevent the chance of falsely judging that there is a clogging in the delivery tube 13 or the return tube 14 when in fact there isn't. The compensation by this time lag can be arbitrarily adjusted in accordance with the length of the delivery tube 13 and the return tube 14.

As can be seen from the above, the present invention makes it possible to detect the possibility of abnormal conditions such as leakage and advise the operator in early stages by making a judgment whether it should advance to the interruption processes (a) through (d) and making such advances as needed either in the refilling mode or in the treatment mode.

The constitutions of the above embodiment can be arbitrarily modified by a person skilled in the art. For example, following variations are possible.

(Variation 1)

Example variation of the cooling water bag

The cooling water bag 8 that inflates as it is filled with water is used as a container for the cooling water in the above embodiment. However, it also can be replaced with a cooling water cassette of a fixed shape that does not inflate or shrink.

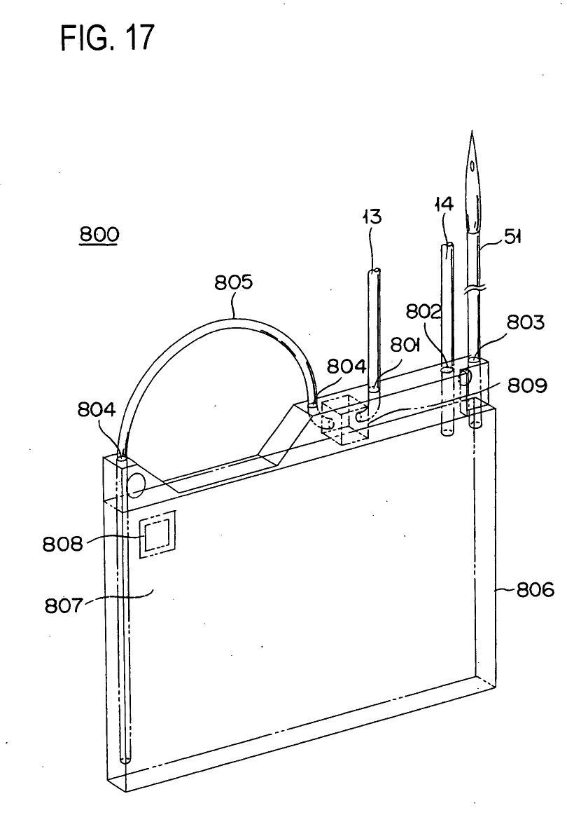

FIG. 17 is a diagram showing an example of a cooling water cassette.

The cooling water cassette 800 is primarily made of a hard material such as poly carbonate. The cooling water cassette 800 is provided on top thereof with a delivery tube port 801, a return tube port 802, a replenishing tube port 803, and a pump tube port 804.

The delivery tube 13 is connected to the delivery tube port 801, the return tube 14 is connected to the return tube port 802, and the refilling tube 51 is connected to the refilling tube port 803.

A pump tube port 805 is mounted on the pump tube port 804. The pump tuber 805 is squeezed by the rotary pump 72 when the cooling water cassette 800 is mounted on the cooling device 7.

At the bottom of the cooling water cassette 800 provided a tank unit 806 for storing the cooling water. The tank unit 806 is provided with a film unit 807 is provided on one side thereof with a large surface. The film unit 807 is not made of a hard material but of a film material. When the cooling cassette 800 is mounted on the cooling device 7, the film unit 807 makes a close contact with the temperature adjusting unit 74. This close contact enables the temperature of the temperature adjusting unit 74 to be transferred efficiently to the cooling water.

An air filter 808 is provided at the top of the tank unit 806. The air filter 808 is a filter for allowing only the air inside the tank unit 806 to pass.

A waterwheel unit 809 is provided in the cooling cassette 800 between the pump tube port 804 and the delivery tube port 801. The internal constitution of the waterwheel unit 809 is identical to that of the waterwheel unit 141 of the abovementioned embodiment.

When the cooling water cassette 800 is attached to the cooling device 7, the pump tube 805 is positioned automatically to contact with the rotary tube 131, and the waterwheel unit 809 is positioned automatically to a position suitable for the detection of the flow sensor 73.

As can be seen from the above, the example variation 1 simplifies the mounting procedure and makes the treatment more efficient as the waterwheel unit 809 is built in so that the waterwheel 809 is automatically position to a position suitable for the detection of the flow sensor 73 as the cooling water cassette 800 is mounted on the cooling device 7.

(Variation 2)

Example variation 2 of the cooling water bag

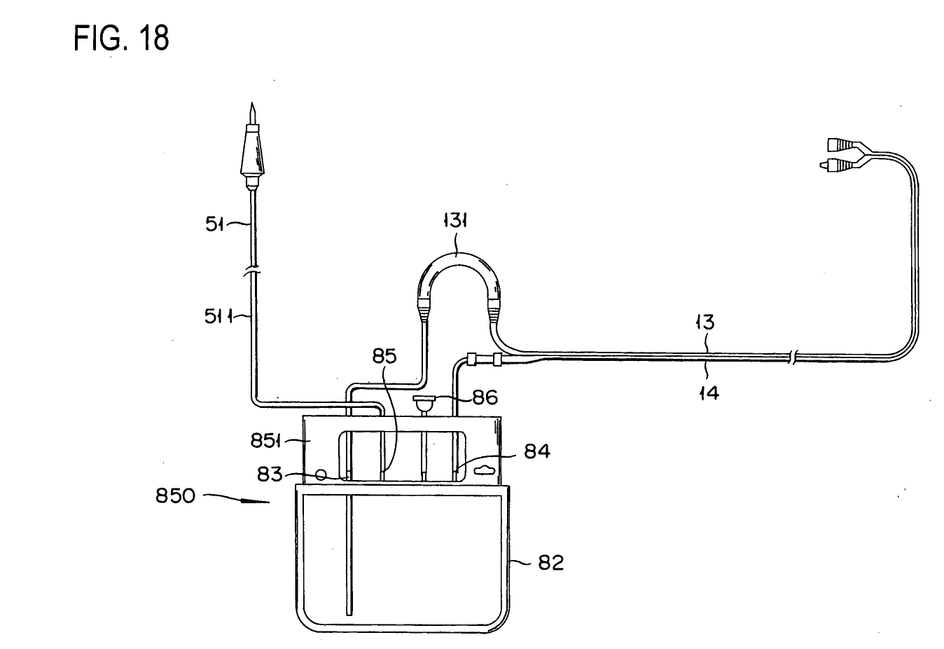

FIG. 18 is a diagram showing another example of a cooling water bag.

The cooling water bag 850 is different from the cooling water bag 8 of the above embodiment in that a hunger 851 serves as the holder's function as well for holding each tube.

The cooling water bag 850 in the variation example 2 has the delivery tube 13, the return tube 14, the refilling tube 51, and the air vent 86 are inserted through the hunger 851 as shown in FIG. 18. Consequently, these tubes are held without drooping.

With this constitution, the cooling water bag 850 can be easily mounted on the cooling device 7 without having to holding those tubes in upright positions.

(Variation 3)

Variation Example of the Closing Part

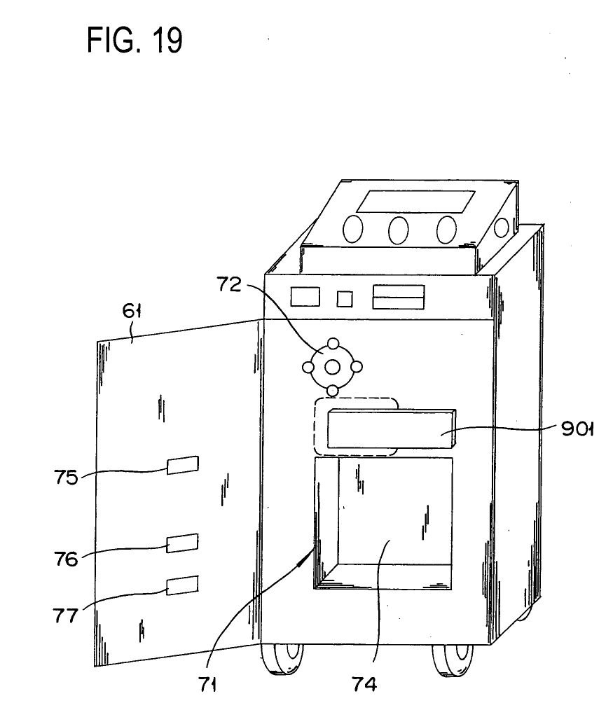

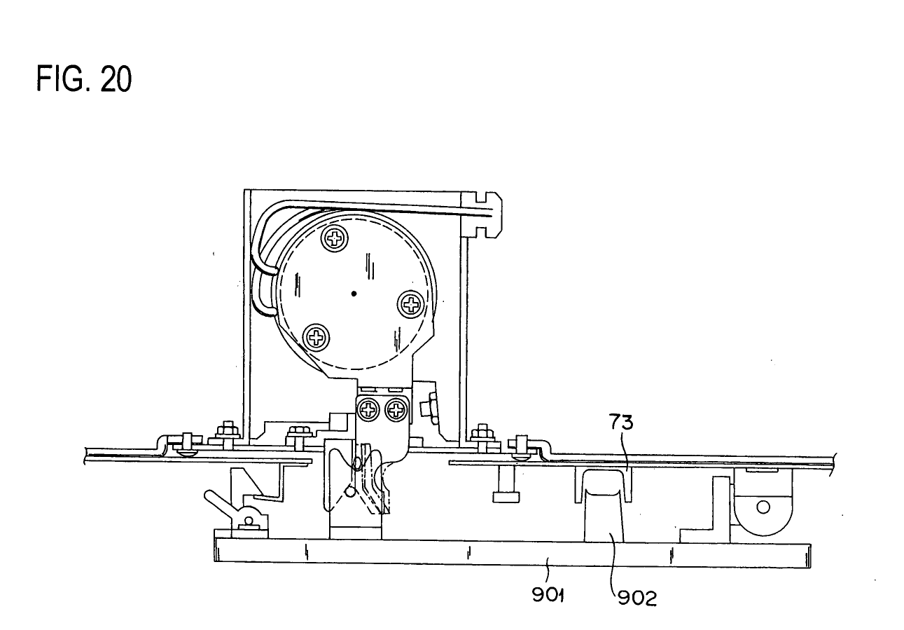

FIG. 19 is a diagram showing the control main unit and FIG. 20 is a diagram showing the closing part.

As shown in FIG. 19, a small door 901 of the closing part 9 is made longer toward the right side of the drawing to cover the flow sensor 73 in the variation example 3. A waterwheel holding piece 902 is provide on the inside of the small door 901. The waterwheel holding piece 902 is mounted on the position that faces the flow sensor 73 when the small door 901 is closed.

By providing the waterwheel holding piece 902 as such, the waterwheel unit 141 is pressed against the waterwheel holding piece 902 when the cooling water bag 8 is mounted on the cooling device 7 and the small door 901 is closed. Therefore, the waterwheel unit 141 is securely mounted on the flow sensor 73.

While the present invention has been particularly shown and described with reference to exemplary embodiments thereof, it will be understood by those of ordinary skill in the art that various changes in form and details may be made therein without departing from the spirit and scope of the present invention as defined by the following claims.

The entire disclosure of Japanese Patent Application Nos. 2004-107667 filed on Mar. 31, 2004 including specification, claims and summary are incorporated therein by reference in its entirely.

1. An apparatus for hyperthermia treatment by emitting body tissue with energy comprising:
an applicator;
a cooling fluid container holding cooling fluid to be circulated through said applicator;
a storage unit for storing said cooling fluid to refill said cooling fluid container;
a sensor for detecting whether said cooling fluid's amount held in said cooling fluid container is more than a preset amount or not;
a refill control unit refilling said cooling fluid container with said cooling fluid from said storage unit when said cooling fluid's amount in said cooling fluid container is less than the preset amount;
a total refill timer counting a total refill time required for refilling said cooling fluid container with said cooling fluid from said storage unit; and
an abnormality notifying unit notifying an abnormality when said total refill time exceeds a preset time.

2. An apparatus for hyperthermia treatment claimed in claim 1 further comprising:
a closing part capable of closing or opening said cooling fluid's passage by closing or opening a tube that communicates between said storage unit and said cooling fluid container, wherein
said refill control unit inhibits refill of said cooling fluid container with said cooling fluid from said storage unit by closing said tube or allows refill of said cooling fluid container with said cooling fluid from said storage unit by opening said tube.

3. An apparatus for hyperthermia treatment claimed in claim 1 further comprising:
a pump provided in a circulation passage and circulating said cooling fluid through said applicator from said cooling fluid container;
a pump timer counting an operating time of the pump; and
a flow sensor provided in a circulation passage between said applicator and said cooling fluid container and measuring a flow volume of said cooling fluid that circulates the passage, wherein
said abnormality notifying unit notifies that there is an abnormality when said pump operating time exceeds a preset time and the flow volume measured by said flow sensor is below a preset volume.

4. An apparatus for hyperthermia treatment claimed in claim 1 further comprising:
a temperature setting unit setting a temperature of said cooling fluid;
a temperature measuring unit measuring a temperature of said cooling fluid;
a temperature adjusting unit contacting said cooling fluid container and cooling or heating said cooling fluid container;
a temperature adjusting operation timer counting time said temperature adjusting unit has been operating; and
a temperature controlling unit controlling said temperature adjusting unit by determining the temperature adjusting unit's target operation rate for cooling or heating based on a difference between the preset temperature of the cooling fluid and the measured temperature and raising the operation rate up to said target operation rate in steps in accordance with the counted time by said temperature adjusting operation timer.

5. An apparatus for hyperthermia treatment claimed in claim 1, wherein
said refill control unit further comprising:
a refill timer for counting time for each refill for refilling said cooling fluid container with said cooling fluid from said storage unit each time when said cooling fluid container's cooling fluid amount becomes less than the preset amount after having been more than the preset amount; and
said abnormality notifying unit notifying abnormality when said refill time exceeds a preset time.

6. An apparatus for hyperthermia treatment by emitting body tissue with energy comprising:
an applicator;
a cooling fluid container holding cooling fluid to be circulated through said applicator;
a storage unit storing said cooling fluid to refill said cooling fluid container;
a sensor detecting whether said cooling fluid's amount held in said cooling fluid container is more than a preset amount or not;
a refill control unit refilling said cooling fluid container with said cooling fluid from said storage unit when said cooling fluid's amount in said cooling fluid container becomes less than the preset amount;
a refill timer counting each refill time required for refilling said cooling fluid container with said cooling fluid from said storage unit; and
an abnormality notifying unit notifying abnormality when said refilling time exceeds a preset time.

7. An apparatus for hyperthermia treatment claimed in claim 6 further comprising:
a closing part capable of closing or opening said cooling fluid's passage by closing or opening a tube that communicates between said storage unit and said cooling fluid container, wherein
said refill control unit inhibiting refill of said cooling fluid container with said cooling fluid from said storage unit by closing said tube or allowing refill of said cooling fluid container with said cooling fluid from said storage unit by opening said tube.

8. An apparatus for hyperthermia treatment claimed in claim 6 further comprising:
a pump provided in a circulation passage and circulating said cooling fluid through said applicator from said cooling fluid container;
a pump timer counting an operating time of the punp; and
a flow sensor provided in a circulation passage between said applicator and said cooling fluid container measuring flow volume of said cooling fluid that circulates the passage, wherein
said abnormality notifying unit notifies that there is an abnormality when said pump operating time exceeds the preset time and the flow volume measured by said flow sensor is below the preset volume.

9. An apparatus for hyperthermia treatment claimed in claim 6 further comprising: a temperature setting unit setting a temperature of said cooling fluid;
a temperature measuring unit measuring a temperature of said cooling fluid;
a temperature adjusting unit contacting said cooling fluid container cooling or heating said cooling fluid container; and
a temperature adjusting operation timer counting time said temperature adjusting unit has been operating;
a temperature controlling unit controlling said temperature adjusting unit by determining the temperature adjusting unit's target operation rate for cooling or heating based on a difference between the cooling fluid's preset temperature and the measured temperature and raising the operation rate up to said target operation rate in steps based on the counted time measured by said temperature adjusting operation timer.

10. An abnormality notifying method for an apparatus for hyperthermia treatment comprising steps of:
refilling a cooling fluid container from a storage unit, wherein said cooling fluid is circulated through an applicator used for emitting body tissue with energy;
counting time to refilling said cooling fluid container with cooling fluid as a first counting step; and
notifying that there is an abnormality when the time counted by said first counting step exceeds a preset time.

11. An abnormality notifying method for an apparatus for hyperthermia treatment claimed in claim 10 further comprising steps of:
detecting whether the cooling fluid's amount in said cooling fluid container is more than a preset amount or not;
refilling said cooling fluid container with said cooling fluid from said storage unit when said cooling fluid's amount in said cooling fluid container is less than the preset amount;
counting a time elapsed since the refilling has started as a second counting step; and
notifying that there is an abnormality when the time measured by said second counting step exceeds a preset time.

12. An abnormality notifying method for an apparatus claimed in claim 10 further comprising steps of:
counting an operating time for operating a pump for circulating cooling fluid to said applicator;
measuring an amount of the cooling fluid returning to said cooling fluid container after circulating said applicator; and
notifying that there is an abnormality when said pump's operating time exceeds a preset time and the flow volume measured by a flow sensor is below a preset amount.

13. An abnormality notifying method for an apparatus for hyperthermia treatment claimed in claim 10 further comprising steps of:
setting said cooling fluid's temperature;
measuring said cooling fluid's temperature;
counting a time elapsed since a temperature adjusting unit has started to operate for adjusting said cooling fluid's temperature; and
controlling said temperature adjusting unit by determining the temperature adjusting unit's target operation rate for cooling or heating based on a difference between the cooling fluid's preset temperature and the measured temperature and raising the operation rate up to said target operation rate in steps based on the operating time of said temperature adjusting unit.
